(19) United States
(12) Patent Application Publication
(10) Pub. No.: US 2026/0216232 A1
(43) Pub. Date: Jul. 30, 2026

(54) SEVUPARIN FOR THE TREATMENT OF CHRONIC KIDNEY DISEASE

(71) Applicant: Modus Therapeutics AB, Stockholm (SE)

(72) Inventors: John Öhd, Nacka (SE); Maura Poli, Brescia (IT)

(21) Appl. No.: 19/141,962

(22) PCT Filed: Dec. 20, 2023
(86) PCT No.: PCT/EP2023/086943

§ 371 Date: Jun. 20, 2025

(30) Foreign Application Priority Data
Dec. 21, 2022 (GB) 2219368.4

(51) Int. Cl. A61K 31/727; A61K 31/437; A61K 31/4418; A61K 31/472; A61K 31/515; A61K 31/5377; A61K 38/18; A61P 7/06; A61P 13/12

(52) U.S. Cl. CPC A61K 31/727; A61K 31/437; A61K 31/4418; A61K 31/472; A61K 31/515; A61K 31/5377; A61K 38/1816; A61P 7/06; A61P 13/12

(57) ABSTRACT

The present invention is directed to the substance sevuparin or a pharmaceutically acceptable salt thereof, for use in the treatment of a subject having a chronic kidney disease (CKD) which may also comprise anemia. This therapy may also be useful as combination therapy with standard of care (SOC) therapy, such as combination therapy with an erythropoiesis-stimulating agent (ESA) or with a HIF prolyl hydroxylase inhibitor agent.

### FIELD OF THE INVENTION

The present invention is directed to the substance sevuparin or a pharmaceutically acceptable salt thereof, for use in the treatment of a chronic kidney disease (CKD) which may also comprise anemia, as well as use of sevuparin in combination therapy with an erythropoiesis-stimulating agent (ESA) or with a HIF prolyl hydroxylase inhibitor agent.

### BACKGROUND OF THE INVENTION

Chronic kidney disease (CKD) is associated with several complications that worsen with progression of disease; anemia, disturbances in iron metabolism and inflammation are common features. The inflammatory response starts early in the disease process, releasing pro-inflammatory cytokines, acute phase reactants and hepcidin. Hepcidin production is modulated by several factors, such as hypoxia/anemia, erythropoietin and erythropoiesis products, transferrin saturation (TSAT) and liver iron levels, which are altered in CKD.

Chronic kidney disease (CKD) may cause anemia. Erythropoiesis-stimulating agents (ESAs), including recombinant erythropoietin (EPO) analogs, have been used for treating anemia associated with Chronic kidney disease (CKD) through compensating for decreased EPO (Thavarajah, S et al: Am. J. Kidney Dis. 2019, 74, pp. 667-674: The Use of Erythropoiesis-Stimulating Agents in Patients With CKD and Cancer: A Clinical Approach). However, resistance and tolerance to these drugs have been shown to develop, and high doses of Erythropoiesis-stimulating agents (ESAs) can also cause side effects such as cardiovascular diseases.

Anemia is a condition in which the blood is unable to deliver sufficient amounts of oxygen to the tissues because of insufficient number of red blood cells (RBCs), which are the oxygen carriers in the blood, and insufficient hemoglobin, which holds molecular oxygen in the red blood cells (RBCs). Anemia symptoms include fatigue, shortness of breath, weakness and dizziness. Iron deficiency is thought to be the most common cause of anemia globally, although other conditions, such as folate, vitamin B12 and vitamin A deficiencies, chronic inflammation, parasitic infections, and inherited disorders can all cause anemia.

Secondary anemia, also called anemia of chronic disease (ACD) or anemia of inflammation, is the commonest form of anemia in hospitalised patients and the second most prevalent anemia worldwide after iron deficiency. It is characterised by defective iron incorporation in erythropoiesis, an impaired response to erythropoietin, a dysregulation in erythropoietin production and cytokine induced shortening of red cell survival. For many patients with ACD the cause is apparent, but for many others the underlying disease needs to be determined and such patients are often referred to haematologists for investigation. In patients with chronic kidney disease (CKD) the marked reduction in erythropoietin production is the most important factor in causing anemia but these patients also have features of ACD (Sarah L. Davis et al; Blood Reviews; Volume 26, Issue 2, March 2012, Pages 65-71).

Erythropoiesis-stimulating agents (ESAs) are medications which stimulate the bone marrow to make red blood cells, thus increasing the oxygen-carrying capacity of the blood. These medications are given by injection and work by stimulating the production of more red blood cells. Erythropoietin (EPO) is a hormone produced by the kidney that promotes the formation of red blood cells by the bone marrow. The kidney cells that make erythropoietin are sensitive to low oxygen levels in the blood that travels through the kidney. These cells make and release erythropoietin when the oxygen level is too low. A low oxygen level may indicate a diminished number of red blood cells (anemia), or hemoglobin molecules that carry oxygen through the body. As the prime regulator of red cell production, erythropoietin's major functions are to promote the development of red blood cells, and to initiate the synthesis of hemoglobin which transports oxygen within red blood cells. Two ESAs on the U.S. market are epoetin alfa (Procrit,® Epogen®), and darbepoietin alfa (Aranesp®).

The production of red blood cells (RBCs) is a coordinated process that requires both the growth factor erythropoietin (EPO) and an adequate iron supply. EPO, produced by the kidney during hypoxia, stimulates the erythroid cells proliferation and differentiation (Franke K et al; Blood 2013; 122 (7) pp. 1122-1128).

To acquire iron, essential for hemoglobin (Hb) synthesis, erythroid precursors release soluble factors that suppress the expression of the hepatic iron regulatory hormone hepcidin in order to increase iron absorption and recycling. Hepcidin is an iron-regulated hepatic peptide hormone that controls iron absorption at the intestinal level, and iron release from macrophages and hepatocytes. Hepcidin binds to the plasma membrane iron exporter ferroportin and induces its endocytosis and degradation, preventing release of iron into the plasma (Nai et al; Blood 12 May 2016; Vol. 127; Number 19; pp. 2327-2336). Hence, increased levels of hepcidin cause retention of iron in macrophages and enterocytes and a reduced availability of iron to erythropoiesis, leading to impaired heme synthesis. Also, hepcidin restricts availability of exogenous iron whether chronically or acutely administered by separate mechanisms (Ramos E et al: Hepatology. 2011 April; 53 (4): pp. 1333-41). Hence, high hepcidin levels counteracts the possibility to take-up iron.

Hepcidin plays a key role in the pathogenesis of ACD, and elevated hepcidin levels have been found in a wide variety of chronic diseases such as inflammatory bowel disease (IBD), infections, myeloma and non-hodgkin lymphoma (Cullis J; Diagnosis and management of anaemia of chronic disease: Current status; Br. J. Haematol 2011:154: pp. 289-300).

Situations with elevated levels of hepcidin are believed to contribute to the inadequate response to EPO in such cases as indicated above as seen in both clinical and experimental studies (Nai A et al. Blood. 2016 May 12; 127 (19): pp. 2327-2336; Steensma D P et al: Blood. 2015 Jun. 4; 125 (23): pp. 3669-3671; Petrulienė K et al: Medicina Kaunas; 2017; 53 (2): pp. 90-100).

### DESCRIPTION OF THE INVENTION

The present invention is directed to the compound sevuparin or a pharmaceutically acceptable salt thereof, for use in the treatment of a chronic kidney disease (CKD).

In a further aspect of the invention, the chronic kidney disease (CKD) may comprise kidney damage such as fibrosis. In yet an aspect of the invention, a subject suffering from a chronic kidney disease (CKD) may also have anemia.

A problem underlying anemia in chronic kidney disease (CKD) is that increased plasma levels of hepcidin causes retention of iron in macrophages and enterocytes and a reduced availability of iron to erythropoiesis, leading to impaired heme synthesis. Normal levels of hepcidin are about 1 to 12 nml/L. During normal erythropoiesis, when there is an adequate nutritional iron supply and an absence of inflammation, systemic iron homeostasis maintains plasma iron levels in the range of 10-30 UM and whole-body iron stores in the range of 0.3-1 g. This is manifested in the iron storage marker ferritin being 27-365 μg/L in males and 13-148 μg/L in females, as well as the soluble transferrin receptor at 0.76-1.76 mg/L, transferrin saturation at 10/15-50/60% females/males and a total iron binding capacity at 30-80 μmol/L depending on age. The main mechanism of iron homeostasis centers on the interaction between the iron regulatory hormone hepcidin, produced by hepatocytes, and ferroportin, which is both the hepcidin receptor and a cellular iron exporter through which iron is transferred to blood plasma. Baseline hepcidin synthesis is controlled by feedback from both iron stores and plasma iron levels. Increased levels of hepcidin cause retention of iron in macrophages and enterocytes and a reduced availability of iron to erythropoiesis, leading to impaired heme synthesis.

Frequently in anemia associated with chronic kidney disease (CKD), inadequate response to endogenous EPO is seen, as evidenced by elevated levels of EPO and the need to increase the dose over time (normal range 2.6-18.5 IU/L) without adequate improvement on anemia parameters such as hemoglobin, hematocrit and reticulocytes which may affect the production of EPO negatively in Chronic Kidney Disease leading to generally low levels of EPO. In such situations, the inadequate response to EPO is manifested by insufficient improvement on anemia parameters such as hemoglobin, hematocrit and reticulocytes in spite of elevated doses of exogenous EPO (i.e. EPO alfa i.v. higher than 200 IU/kg/week or equivalent).

### BRIEF DESCRIPTION OF THE DRAWINGS

FIG. 1A is a graph showing the hemoglobin blood levels in mice treated with sevuparin for 3 weeks.

FIG. 1B is a graph showing hematocrit in mice treated with sevuparin for 3 weeks.

FIG. 1C is a graph showing body weight in mice treated with sevuparin for 3 weeks.

FIG. 1D is a graph showing serum creatinine levels in mice treated with sevuparin for 3 weeks.

FIG. 1E is a graph showing the blood level of reticulocyte-hemoglobin (Ret-He) in mice treated with sevuparin for 3 weeks.

FIG. 1F is a graph showing the serum level of hepcidin in mice treated with sevuparin for 3 weeks.

FIG. 1G is a graph showing histochemistry and immunostaining on paraffin-embedded kidney sections, illustrating the physical status of mouse kidneys after 3 weeks treatment with sevuparin.

FIG. 2A shows micrographs illustrating the physical status of the mouse kidneys after 11 weeks of treatment with sevuparin, EPO, or a combination of sevuparin and EPO.

FIG. 2B are macroscopic observations (40× magnification) of kidney damage of the organ after 11 weeks treatment with sevuparin, EPO, or a combination of sevuparin and EPO.

FIG. 3A is a graph showing the serum level of hepcidin at 3 weeks treatment with sevuparin, EPO, or a combination thereof.

FIG. 3B is a graph showing the serum level of hepcidin at 6 weeks treatment with sevuparin, EPO, or a combination thereof.

### DETAILED DESCRIPTION OF THE INVENTION

An object of the present invention is the compound sevuparin or a pharmaceutically acceptable salt thereof, for use in the treatment of a chronic kidney disease (CKD).

An aspect of the present invention is the compound sevuparin which has:
(i) an antifactor IIa activity of up to 10 IU/mg;
(ii) an antifactor Xa activity of up to 10 IU/mg;
(iii) a weight average molecular weight from 6.5 to 9.5 kDa;
(iv) wherein the polysaccharide chains in said chemically modified heparin contain a predominant structure of the formula:

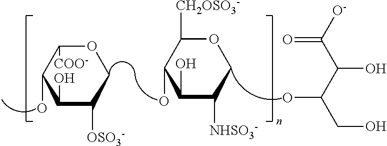

wherein n is an integer of from 2 to 25, such that it comprises from 2 to 25 disaccharide units corresponding to molecular weights from 1.2 to 15 kDa;
(v) retain at least 90% of the sulfate groups compared to native heparin;
(vi) have a reduction in chemically intact pentasaccharide sequences, responsible for providing an antithrombin mediated anticoagulant effect, compared to the polysaccharide chains of native heparin; and
(vii) have a reduction in unsulfated iduronic acid units and glucuronic acid units compared to native heparin;
for use in the treatment of a chronic kidney disease (CKD).

In a further aspect, sevuparin for use as herein described and claimed, has in a 1H-NMR spectrum, no unidentified signals in the ranges 0.10-2.00 ppm, 2.10-3.10 ppm and 5.70-8.00 ppm larger than 4 percent when compared to the height of the signal present in native heparin at 5.42 ppm.

In yet an aspect of the invention, the predominantly occurring polysaccharide chains in the sevuparin as used in accordance with the invention, have from 6 to 16 disaccharide units with molecular weights from 3.6 to 9.6 kDa.

In yet an aspect of the invention, sevuparin as used in accordance with the invention, comprises glycol-split residues of the chemical structure:

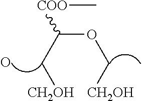

In yet an aspect of the invention, in sevuparin as used in accordance with the invention, at least 30% of the polysaccharide chains have a molecular weight of at least 8 kDa.

In yet an aspect of the invention, in sevuparin as used in accordance with the invention, 3-15% of the polysaccharide chains have a molecular weight of at least 15 kDa.

In yet an aspect of the invention, in sevuparin as used in accordance with the invention, 25-47% of the polysaccharide chains have a molecular weight of at least 9 kDa.

In yet an aspect of the invention, in sevuparin as used in accordance with the invention, 40-60% of the polysaccharide chains have a molecular weight of at least 7 kDa.

In yet an aspect of the invention, in sevuparin as used in accordance with the invention, 60-80% of the polysaccharide chains have a molecular weight of at least 5 kDa.

In yet an aspect of the invention, in sevuparin as used in accordance with the invention, at least 85% of the polysaccharide chains have a molecular weight of at least 3 kDa.

In yet an aspect of the invention, in sevuparin as used in accordance with the invention, at least 95% of the polysaccharide chains have a molecular weight of at least 2 kDa.

An aspect of the present invention is the compound sevuparin for use in a chronic kidney disease (CKD) as herein described, wherein said chronic kidney disease (CKD) comprises kidney damage.

An aspect of the present invention is the compound sevuparin for use in a chronic kidney disease (CKD) as herein described, wherein the kidney damage comprises inflammation.

An aspect of the present invention is the compound sevuparin for use in a chronic kidney disease (CKD) as herein described, wherein the chronic kidney disease (CKD) comprises fibrosis.

An aspect of the present invention is the compound sevuparin for use in a chronic kidney disease (CKD) as herein described, wherein the chronic kidney disease (CKD) comprises an impaired kidney function with a CKD glomular filtration rate (GFR) which is G2, G3a, G3b, or G4, as defined by the National Kidney Foundation.

An aspect of the present invention is the compound sevuparin for use in a chronic kidney disease (CKD) as herein described, wherein a subject suffering from the chronic kidney disease (CKD) as herein described is at risk of developing kidney failure.

An aspect of the present invention is the compound sevuparin for use in a chronic kidney disease (CKD) as herein described, wherein the subject suffering from the chronic kidney disease (CKD) has kidney failure.

An aspect of the present invention is the compound sevuparin for use in a chronic kidney disease (CKD) as herein described, wherein the subject suffering from the chronic kidney disease (CKD) also has anemia.

An aspect of the present invention is the compound sevuparin for use in a chronic kidney disease (CKD) as herein described, wherein the anemia is anemia in chronic disease (ACD).

An aspect of the present invention is the compound sevuparin for use as herein described, wherein the anemia is renal anemia.

An aspect of the present invention is the compound sevuparin for use in a chronic kidney disease (CKD) as herein described, wherein the sevuparin is used in combination therapy with an agent for use as standard of care (SOC) therapy in a chronic kidney disease (CKD).

One aspect of the invention, is sevuparin for use as monotherapy.

In one aspect of the present invention, the compound sevuparin is used in combination therapy with an erythropoiesis-stimulating agent (ESA) as the standard of care (SOC) agent. An example of an erythropoiesis-stimulating agent (ESA) which may be used in combination therapy with sevuparin as herein described, is an erythropoietin (EPO). Examples of such erythropoietins (EPOs) may be selected from any one of erythropoietin alfa; erythropoietin beta; erythropoietin epsilon; erythropoietin gamma; erythropoietin kappa; erythropoietin omega; erythropoietin theta; and erythropoietin zeta.

In yet an aspect of the present invention, the compound sevuparin may be used in combination therapy with a HIF prolyl hydroxylase inhibitor as the standard of care (SOC) agent. Examples of such HIF prolyl hydroxylase inhibitor may be selected from Roxadustat, Vadadustat, Dapurodustat, Enarodustat, and Molidustat.

In one aspect of the invention, sevuparin is for use as add-on therapy, wherein sevuparin is administered to a subject suffering from a chronic kidney disease (CKD) as herein described and which subject is already being treated with a standard of care (SOC) agent indicated for therapy of a chronic kidney disease (CKD).

In one aspect of the invention, sevuparin is co-administered with a standard of care (SOC) agent indicated for therapy of a chronic kidney disease (CKD).

In one aspect of the invention, sevuparin and a standard of care (SOC) agent is administered simultaneously, separately or sequentially in therapy of a chronic kidney disease (CKD).

One aspect of the invention, is the compound sevuparin for use in the treatment of a chronic kidney disease (CKD) as herein described, wherein monotherapy with an erythropoiesis-stimulating agent (ESA) does not provide adequate therapeutic effect.

One aspect of the invention, is the compound sevuparin for use in lowering the level of hepcidin in the blood of a subject suffering from anemia.

One aspect of the invention, is the compound sevuparin for use in lowering the level of hepcidin in the blood of a subject suffering from a chronic kidney disease.

Yet an aspect of the invention, is the compound sevuparin for use in lowering the level of hepcidin in the blood of a subject suffering from a chronic kidney disease and which subject has anemia.

One aspect of the invention, is the compound sevuparin for use in the treatment of a chronic kidney disease (CKD) as herein described, wherein the therapeutic effect of sevuparin does not depend on the serum level of hepcidin in the subject being treated.

One aspect of the invention, is the compound sevuparin for use in the treatment of a chronic kidney disease (CKD) as herein described, wherein said use is for restoring responsiveness to erythropoietin (EPO) in a subject having a chronic kidney disease (CKD) with anemia. Renal anemia is a common complication of hemodialysis patients. Erythropoietin (EPO) hyporesponsiveness has been recognized as an important factor to poor efficacy of recombinant human erythropoietin in the treatment of renal anemia. More importantly, increased erythropoiesis resistance index (ERI) may be associated with inflammation and increased mortality (Xiangxue Lu et al; Hindawi Mediators of Inflammation Volume 2020, Article ID 1027230).

ESA resistance or hyporesponsiveness occurs when the patient does not reach the desired serum hemoglobin (Hb) concentration even with the use of ESA at doses higher than usual or when increasingly higher doses are necessary to maintain the recommended Hb concentration (Drüeke T B et al: Summary of the KDIGO guideline on anemia and comment: reading between the (guide)line(s). Kidney Int. 2012; 82 (9): pp. 952-960).

Weiner et al (J Am Soc Nephrol 18: pp. 3184-3191, 2007 Reducing versus discontinuing erythropoietin at high hemoglobin levels) has suggested that discontinuation, rather than reduction, of ESA treatment may be more appropriate when hemoglobin level reaches 130 g/L or above. When hemoglobin levels are higher than this, the patient risk increases as described above and may also be at risk of erythroid polycythemia. Erythroid polycythemia is defined as a hemoglobin value of 165 g/L for men and 160 g/L for women.

One aspect of the invention, is the compound sevuparin for use in the treatment of a chronic kidney disease (CKD) as herein described, wherein the subject suffering from said chronic kidney disease (CKD) is an elderly subject.

One aspect of the invention, is the compound sevuparin for use in the treatment of a chronic kidney disease (CKD) as herein described, wherein said use is for increasing the blood level of hemoglobin.

Yet an aspect of the invention is the use of the compound sevuparin for the manufacture of a medicament for the treatment of a chronic kidney disease (CKD) as herein described and claimed.

Yet an aspect of the invention is a method for the treatment of a chronic kidney disease (CKD), comprising administering a therapeutically effective amount of the compound sevuparin or a pharmaceutically acceptable salt thereof, to a subject in need of such treatment.

### Definitions

Sevuparin is a heparin derivative with the company compound code DF02. The INN (International Nonproprietary Name) for DF02 is sevuparin sodium. The CAS registry number (RN) for sevuparin is RN 9041-08-1.

More specifically, sevuparin is a chemically modified heparin having:
(i) an antifactor 11a activity of up to 10 IU/mg;
(ii) an antifactor Xa activity of up to 10 IU/mg;
(iii) a weight average molecular weight from 6.5 to 9.5 kDa;
(iv) wherein the polysaccharide chains in said chemically modified heparin contain a predominant structure of the formula:

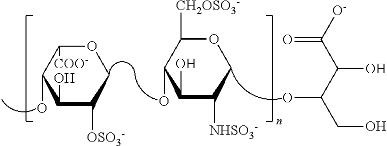

wherein n is an integer of from 2 to 25, such that it comprises from 2 to 25 disaccharide units corresponding to molecular weights from 1.2 to 15 kDa;
(v) retain at least 90% of the sulfate groups compared to native heparin;
(vi) have a reduction in chemically intact pentasaccharide sequences, responsible for providing an antithrombin mediated anticoagulant effect, compared to the polysaccharide chains of native heparin; and
(vii) have a reduction in unsulfated iduronic acid units and glucuronic acid units compared to native heparin.

Sevuparin may have in a 1H-NMR spectrum, no unidentified signals in the ranges 0.10-2.00 ppm, 2.10-3.10 ppm and 5.70-8.00 ppm larger than 4 percent when compared to the height of the signal present in native heparin at 5.42 ppm.

The predominantly occurring polysaccharide chains in sevuparin, have from 6 to 16 disaccharide units with molecular weights from 3.6 to 9.6 kDa, and may comprise glycol-split residues of the chemical structure:

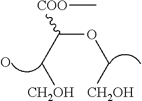

At least 30% of the polysaccharide chains of the compound sevuparin have a molecular weight of at least 8 kDa, 3-15% of the polysaccharide chains have a molecular weight of at least 15 kDa, 25-47% of the polysaccharide chains have a molecular weight of at least 9 kDa, 40-60% of the polysaccharide chains have a molecular weight of at least 7 kDa, 60-80% of the polysaccharide chains have a molecular weight of at least 5 kDa, at least 85% of the polysaccharide chains have a molecular weight of at least 3 kDa, and at least 95% of the polysaccharide chains have a molecular weight of at least 2 kDa.

The mean molecular weight of the major disaccharide is about 600 Daltons.

The manufacture of sevuparin is described in Examples 1 to 3 of the published patent application WO 2013/095276.

The wording chronic kidney disease (CKD) means decreased kidney function shown by a glomerular filtration rate (GFR) of less than 60 ml/min per 1.73 m², or markers of kidney damage, or both, of at least 3 months duration, regardless of the underlying cause.

GFR (glomerular filtration rate) is equal to the total of the filtration rates of the functioning nephrons in the kidney. A rise in blood creatinine levels is observed only after significant loss of functioning nephrons, and GFR is considered the optimal way to measure kidney function. Normal GFR varies according to age, sex, and body size. GFR is usually estimated from the person's serum creatinine and/or cystatin C level, in combination with demographic factors such as age, race, and gender using an estimating equation. The National Kidney Foundation has issued defined categories for the stages and severity of chronic kidney disease (CKD), as set out in Table 1 below.

TABLE A
GFR Categories in CKD

| Cate- | | | |
|---|---|---|---|
| gory | GFR | Terms | Clinical Presentations |
| G1 | ≥90 | Normal or high | Markers of kidney damage (nephrotic |
| | | | syndrome, nephritic syndrome, |
| | | | tubular syndromes, urinary tract |
| | | | symptoms, asymptomatic urinalysis |
| G2 | 60-89 | Mildly decreased* | abnormalities, asymptomatic |
| | | | radiologic abnormalities, |
| | | | hypertension) due to kidney disease. |
| G3a | 45-59 | Mildly to moderately decreased | Mild to severe complications: |
| | | | Anemia; mineral and bone disorder |
| G3b | 30-44 | Moderately to severely decreased | such as elevated parathyroid |
| G4 | 15-29 | Severely decreased | hormone; cardiovascular disease such |
| | | | as hypertension, lipid abnormalities, |
| | | | low serum albumi. |
| G5 | <15 | Kidney failure | Includes all of the above AND |
| | | | Uremia. |

GFR = ml/min/1.73 m²
*Relative to young adult level

In the absence of evidence of kidney damage, neither GFR category G1 nor G2 fulfill the criteria for CKD. When GFR<30 ml/min/1.73 m² a nephrologist may have to prepare for kidney replacement.

The wording kidney damage as used herein means the presence of asymptomatic and symptomatic markers of Kidney damage according to the classification described by The National Kidney Foundation (see table 1 above), including markers present in blood analyses as well as damage indicators present in the kidney and kidney tissue

The wording inflammation as used herein means the normal or maladaptive or pathological response of an organism to harmful stimuli as evidenced by measurement of, or observation of, at least one agreed marker of inflammation either systemically, in tissue or both. Maladaptive and pathological inflammation are often involved in processes leading to more permanent damage such as but not limited to fibrosis.

The wording fibrosis as used herein is the pathological process whereby functional tissue as a result of repeated injuries, chronic inflammation and repair is replaced by connective scar forming tissue.

The wording kidney failure as used herein means a medical condition in which the kidneys can no longer adequately filter waste products from the blood, functioning at less than 15% of normal levels. Kidney failure is classified as acute kidney failure which develops rapidly and may resolve; and chronic kidney failure, which develops slowly and can often be irreversible. Kidney failure is also known as end-stage kidney disease and is of category G5 according to The National Kidney Foundation.

The wording anemia as used herein, is a condition in which the blood is unable to deliver sufficient amounts of oxygen to the tissues because of insufficient number of red blood cells (RBCs), which are the oxygen carriers in the blood, and insufficient hemoglobin, which holds molecular oxygen in the red blood cells (RBCs). Anemia symptoms include fatigue, shortness of breath, weakness and dizziness. Iron deficiency is thought to be the most common cause of anemia globally, although other conditions, such as folate, vitamin B12 and vitamin A deficiencies, chronic inflammation, parasitic infections, and inherited disorders can all cause anemia.

Secondary anemia, also called anemia of chronic disease (ACD) or anemia of inflammation, is characterised by defective iron incorporation in erythropoiesis, an impaired response to erythropoietin, a dysregulation in erythropoietin production and cytokine induced shortening of red cell survival. In patients with chronic kidney disease (CKD) the marked reduction in erythropoietin production is the most important factor in causing anemia but these patients also have features of the ACD (Sarah L. Davis et al; Blood Reviews; Volume 26, Issue 2, March 2012, Pages 65-71).

The wording renal anemia means anemia of chronic kidney disease (CKD), i.e. a subject suffering from a chronic kidney disease and who also suffers from anemia, has renal anemia.

The wording treatment or therapy as used herein takes the normal wording within the medical and pharmaceutical field, and includes therapeutic treatment as well as prophylactic (preventive) treatment.

Whenever the wording therapeutic treatment is used herein, it means treatment of a subject suffering from a chronic kidney disease (CKD) which may also comprise anemia, as herein described. This treatment (therapy) may be monotherapy with sevuparin alone, or combination therapy with sevuparin and a standard of care (SOC) agent for use in the treatment of a chronic kidney disease such as an erythropoiesis-stimulating agent (ESA) and/or a HIF prolyl hydroxylase inhibitor.

Whenever the wording prophylactic therapy, prophylactic treatment or preventive treatment is used herein, it means that sevuparin is used in a subject at risk of being diagnosed with a chronic kidney disease (CKD) or at risk of developing kidney failure, which may also comprise anemia as herein disclosed and claimed. This treatment (therapy) may be monotherapy with sevuparin alone, or combination therapy with sevuparin and an erythropoiesis-stimulating agent (ESA) and/or a HIF prolyl hydroxylase inhibitor.

The wording monotherapy as used herein, means therapy of a chronic kidney disease (CKD) which may also comprise anemia, as herein described, with the compound sevuparin alone. Monotherapy may be therapeutic therapy (treatment) or preventive therapy (prophylactic therapy).

The wording combination therapy as used herein, means therapy of a chronic kidney disease (CKD) which may also comprise anemia, as herein described, wherein the compound sevuparin and a standard of care (SOC) agent for use in the treatment of a chronic kidney disease such as an erythropoiesis-stimulating agent (ESA) and/or a HIF prolyl hydroxylase inhibitor are used in combination. Such combination therapy may be add-on therapy, co-administration, simultaneous administration or sequential administration.

The wording add-on therapy as used herein, is defined as combination therapy wherein the compound sevuparin is administered to a subject who is already being treated with a standard of care (SOC) agent for use in the treatment of a chronic kidney disease which may also comprise anemia, as herein described, such as an erythropoiesis-stimulating agent (ESA) and/or a HIF prolyl hydroxylase inhibitor.

The wording simultaneous administration as used herein, means that sevuparin and a standard of care (SOC) agent for use in the treatment of a chronic kidney disease which may also comprise anemia, as herein described, such as an erythropoiesis-stimulating agent (ESA) and/or a HIF prolyl hydroxylase inhibitor, are administered simultaneously.

The wording co-administration as used herein, means that sevuparin and a standard of care (SOC) agent for use in the treatment of a chronic kidney disease which may also comprise anemia, as herein described, such as an erythropoiesis-stimulating agent (ESA) and/or a HIF prolyl hydroxylase inhibitor, are administered at the same time, i.e. administered separately from each other but as combination therapy, to the subject being treated.

The wording sequental administration as used herein, means that the compound sevuparin is administered prior to or after administration of a standard of care (SOC) agent for use in the treatment of a chronic kidney disease which may also comprise anemia, as herein described, such as an erythropoiesis-stimulating agent (ESA) and/or a HIF prolyl hydroxylase inhibitor.

The wording less than normal haemoglobin (low blood count) is defined according to WHO. According to the World Health Organization (WHO), anemia is defined as hemoglobin (Hb) levels <12.0 g/dL in women and <13.0 g/dL in men. However, normal Hb distribution varies not only with sex but also with ethnicity and physiological status. New lower limits of normal Hb values have been proposed, according to ethnicity, gender, and age. For the classification and diagnosis the hematologic parameters, the underlying pathological mechanism and patient history should be taken into account. The aging of population, especially in Western countries, causes an increase of anaemia in elderly people. In this population, anaemia, recently defined by levels of Hb<12 g/dL in both sexes, is mostly of mild degree (10-12 g/dL).

The wording Hepcidin-related anemia as used herein means anemia where the serum levels of hepcidin, ferritin, total iron-binding capacity (TIBC); transferrin saturation; soluble transferrin-receptor are abnormal according to a pattern that indicates the presence of so called functional iron deficiency anemia which is synonymous to the terms anemia of chronic disease and anemia of inflammation.

Hepcidin is a protein that in humans is encoded by the HAMP gene. Hepcidin is a key regulator of the entry of iron into the circulation in mammals. In medical conditions in which the hepcidin level is abnormally high, serum iron falls due to iron trapping within macrophages and liver cells and decreased gut iron absorption. This typically leads to anemia due to an inadequate amount of serum iron being available for developing red blood cells.

Erythropoiesis is defined as the process which produces red blood cells. It is stimulated by decreased oxygen in circulation, which is detected by the kidneys, which then secrete the hormone erythropoietin. This hormone stimulates proliferation and differentiation of red cell precursors, which activates increased erythropoiesis in the hemopoietic tissues, ultimately producing red blood cells (erythrocytes).

Erythropoiesis-stimulating agents (ESAs) such as exogenous erythropoietin, i.e recombinant human erythropoietin (rhEPO), are agents produced by recombinant DNA technology in cell culture. Epoetin alfa (Epogen) is a 165-amino acid erythropoiesis-stimulating glycoprotein produced in cell culture using recombinant DNA technology and is used for the treatment of patients with anaemia associated with various clinical conditions. It has a molecular weight of approximately 30,400 daltons and is produced by mammalian cells into which the human erythropoietin gene has been introduced. The product contains the identical amino acid sequence of isolated natural erythropoietin and has the same biological activity as the endogenous erythropoietin.

Erythropoietin (EPO) is a growth factor produced in the kidneys that stimulates the production of red blood cells. It works by promoting the division and differentiation of committed erythroid progenitors in the bone marrow.

Examples of Erythropoiesis-stimulating agents (ESAs) that may be useful in combination therapy as herein described and claimed are epoetin alfa (Procrit,® Epogen®) and darbepoietin alfa (Aranesp®); erythropoietin beta; erythropoietin epsilon; erythropoietin gamma; erythropoietin kappa; erythropoietin omega; erythropoietin theta; and erythropoietin zeta.

The wording Hypoxia-inducible factor (HIF) prolyl hydroxylase inhibitor means a compound that restores EPO production, and which may also optimize iron metabolism by reducing hepcidin levels. Roxadustat, vadadustat, dapurodustat, enarodustat, and molidustat.

The wording ARD means adenine rich diet.

The wording therapeutic effect of sevuparin which does not depend on the serum level of hepcidin, means that a relevant treatment result using sevuparin as described herein, can be achieved without any correlated change, such as lowering of the measured blood levels of hepcidin.

The wording use for restoring, enhancing or potentiating responsiveness to erythropoietin (EPO) in a subject having anemia, means that a desired effect by treatment with EPO is improved or relieved by the addition of treatment with another pharmaceutical compound such as sevuparin (i.e as a combination therapy).

The level of reticulocytes is defined as the level of hemoglobin (Hb) in the reticulocytes. For a subject having hematopiesis, reticulocyte-hemoglobin (Ret-He) is one of the most sensitive variables measuring the efficiency of haemoglobin and red cell formation. It reflects the amount of functional iron available to erythropoiesis in the near term, meaning that a high level may indicate favourable circumstances for achieving efficient erythropoiesis whereas a low level may indicate the opposite Hence, even a modest increase or decrease of Ret-He may have a large impact on the subsequent Hb level and consequently the success of an anemia treatment (Auerbach M et al 2015 Mayo Clin Proc. 2021; 96 (6): pp. 1510-1519; Mast E et al 2008: Am J of Hematol; 83: pp. 307-310).

### Pharmaceutical Formulations and Administration Routes

The compound sevuparin as used in accordance with the present invention, may be administered as a pharmaceutical formulation. Suitable administration routes are systemically by parenteral administration, such as subcutaneous administration, intravenous injection or infusion.

A further aspect of the invention, is oral administration of the compound sevuparin, when used in accordance with the present invention.

For parenteral administration, the compound sevuparin, may be incorporated into a solution or suspension, which may also contain one or more adjuvants such as sterile diluents such as water for injection, saline, fixed oils, polyethylene glycol, glycerol, propylene glycol or other synthetic solvents, antibacterial agents, antioxidants, chelating agents, buffers and agents for adjusting the osmolality. The parenteral preparation may be delivered in ampoules, vials, prefilled or disposable syringes also for self administration, or as infusion arrangements, such as for intravenous or subcutaneous infusion.

Pharmaceutical compositions useful in anaemia therapy according to the invention, may comprise sevuparin, together with at least one conventional pharmaceutically and pharmacologically acceptable excipient and/or carrier. The excipient and/or carrier may be a solid, semisolid or liquid material that can serve as a vehicle for the active substance.

### EXAMPLES

### Manufacture of Sevuparin

Sevuparin, as used in accordance with the present invention, may be prepared by following the synthetic procedure as described in Examples 1 to 3 of the published patent application WO 2013/095276.

In the experiments performed below, sevuparin was provided by Modus Therapeutics AB, Sweden, as a 150 mg/ml solution.

### Biological Evaluation

### Kidney Disease and Anemia of Kidney Disease (KD) Mouse Model

All experimental procedures were approved by the Animal Care and Use Committee of University of Brescia. All animals were maintained on normal diet (“Chow” or Standard diet, 4RF21, obtained from Mucedola s.r.l) if not specified otherwise as part of the “Anemia of Chronic Kidney Disease (CKD) mouse model” experiment as described below.

Animals. The C57BL/6J male mice were purchased from Envigo RMS S.r.l.

Kidney damage, kidney disease and anemia were induced by an adenine high phosphorus diet denoted as “adenine rich diet” (ARD) (0.2% Adenine; 0.9% Phosphorus; 0.6% Calcium; 20% Casein; Cod. S1102-E750 (purchased from Charles River, Manufacturer: Ssniff Spezialdiäten GmbH).

ARD was fed to C57BL/6J male mice having reached an age of five-weeks. The treatments, reported below, started after 5.5 and 9 weeks of ARD at which time the animals developed moderate and severe signs respectively of kidney damage as manifested by weight loss, anemia (defined as a hemoglobin level which had decreased to approximately ≤12.5 g/dL or lower) and by increased serum creatinine levels, as well as kidney tissue damage assessed by macro- and microscopic inspection. All animals were maintained on ARD or normal diet (Standard diet, 4RF21, Mucedola s.r.l), at timings and extents described in detail for each experiment below. Groups of mice were also kept under normal diet (Standard diet) as healthy control group, for each experiment. Mice had access to food and diet ad libitum.

### Summary of Treatments

Adenine rich diet (ARD) (0.2% Adenine; 0.9% Phosphorus; 0.6% Calcium; 20% (Ssniff Spezialdiäten GmbH), sevuparin 10 mg/kg (Opocrin S.p.A., Apotek Produktion & Laboratorier AB, subcutaneously administered, sc), erythropoietin (EPO, Darbepoietin alfa, Aranesp, AMGEN INC.) intraperitoneally adminstered, ip)

### Blood and Serum Analysis.

The hematological parameters hemoglobin (Hb) and hematocrit (Ht) were analyzed using the Hemo Vet Instrument (Infratech) by collecting a single drop of blood from the dorsal pedal vein. The animals weight were monitored weekly in the morning at the following time points of treatment: At start of treatment (P0); after one week of treatment (T1); after two weeks of treatment (T2); after three weeks of treatment (T3); after four weeks of treatment (T4); after five weeks of treatment (T5); after six weeks of treatment (T6); after seven weeks of treatment (T7); after eight weeks of treatment (T8); and after nine weeks of treatment (T9).

Reticulocyte hemoglobin content (Ret-He), serum hepcidin and serum creatinine (analysis performed by Izler Institute) were analyzed at the end of each experiment)

Mice were sacrificed at the indicated time points and blood and kidneys were collected for analysis at the end-point of each experiment.

At the end-point, mouse serum hepcidin was quantified using ELISA kit (Cod. SKU #HMC-001; Intrinsic LifeScience).

Serum creatinine and blood Reticulocytes-hemoglobin (Ret-He) were analyzed by IZLER Institute (Istituto Zooprofilattico Sperimentale della Lombardia e Dell′Emilia Romagna), using standard procedures.

### Histochemistry and Immunostaining on Paraffin-Embedded Kidney Sections

Kidney samples were formalin-fixed and paraffin-embedded. Representative sections were selected based on adequate tissue preservation, as assayed by hematoxylin and eosin (H&E) staining.

Kidney fibrosis was assessed using Sirius Red staining, for collagen deposition, using a standard procedure Using thelmagel software (Schneider, C. A., Rasband, W. S., & Eliceiri, K. W. (2012). NIH Image to ImageJ: 25 years of image analysis. Nature Methods, 9 (7), 671-675. doi:10.1038/nmeth.2089), and with methodology as reported by Ruifrok A C. et al. 2001 (Quantification of histochemical staining by color deconvolution; Anal Quant Cytol Histol. 2001; 23 (4): pp. 291-299; and Landini G. et al. 2021 (Colour deconvolution: stain unmixing in histological imaging. Bioinformatics. 2021; 37 (10): pp. 1485-1487).

Sections were de-waxed, re-hydrated and rinsed in distilled water. Then the slides were submerged in Sirius Red solution (Picric Acid Solution and Sirius Red F33A-CROMA 1A 280) for 50 minutes, rinsed with tap water and counterstained with Mayer Hematoxylin. Sections were then washed in tap water, dehydrated and mounted.

Images were acquired with Nikon DS-Ri2 camera (4908×3264 full-pixel) mounted on Nikon Eclipse 50i microscope equipped with Nikon Plan lenses using NIS-Elements imaging software 4.3 (Nikon Corporation) at 20× or 40× magnification.

Kidneys (1 kidney/mouse) were stained with Hematoxylin and Eosin for routine examination and with Sirius Red for collagen analysis. Four representative images and quantification of Sirius Red staining was performed using imageJ software.

Four images (4 separate fields) from each kidney were acquired at 20× Magnification. The quantification of each field was performed using the ImageJ software, and the means of the 4 fields/mouse were analyzed and reported using GraphPad as two mean values (one for each kidney analysed). Data are represented as the % of positive area to the staining.

Test results (data) are shown as dot plots, block charts and data tables, with Mean±SD.

### Treatment Regimens in Mice Exposed to ARD

### Example 1

ARD Pretreatment for 9 Weeks Followed by 3 Weeks Treatment with Either Control or Sevuparin

C57BL/6J male mice were exposed to ARD pretreatment for 9 weeks followed by 3 weeks of randomized allocation to treatments as defined below still with maintained ARD exposure. One group of mice was given normal diet (no ARD) for the entire experiment, as healthy control.
a) Treatment with control: mice treated with PBS s.c. every day for 3 weeks.
b) Treatment with sevuparin: mice treated with 10 mg/kg Sevuparin s.c. every day for 3 weeks.

### Results

The hematological parameters Hemoglobin (Hb), hematocrit (Ht), weight, creatinine, reticulocyte-hemoglobin and hepcidin were analyzed, and are summarized in Table 1A below.

TABLE 1A

| | | ARD-PBS | |
|---|---|---|---|
| | Normal diet | (control) | ARD-SEV |
| | N = 3 | N = 2 | N = 3 |
| Hb [g/dL] | 15.87⁽¹⁾ | 8.75⁽²⁾ | 10.80⁽³⁾ |
| Ht [%] | 46.67⁽⁴⁾ | 25.00⁽⁵⁾ | 31.67⁽⁶⁾ |
| Wt [g] | 27.23⁽⁷⁾ | 15.00⁽⁸⁾ | 15.00⁽⁹⁾ |
| S-Crea [mg/dL] | 0.60⁽¹⁰⁾ | 0.82⁽¹¹⁾ | 0.73⁽¹²⁾ |
| Ret-He [pg] | 17.53⁽¹³⁾ | 13.1⁽¹⁴⁾ | 14.33⁽¹⁵⁾ |
| S-Hepcidin [nM] | 21.71⁽¹⁶⁾ | 46.85⁽¹⁷⁾ | 36.83⁽¹⁸⁾ |

⁽¹⁾SD = 1.00; ⁽²⁾SD = 0.35; ⁽³⁾SD = 0.44;
⁽⁴⁾SD = 3.06; ⁽⁵⁾SD = 1.41; ⁽⁶⁾SD = 1.15;
⁽⁷⁾SD = 3.44; ⁽⁸⁾SD = 0.71; ⁽⁹⁾SD = 0.71;
⁽¹⁰⁾SD = 0.03; ⁽¹¹⁾SD = 0.00080; ⁽¹²⁾SD = 0.05;
⁽¹³⁾SD = 0.39; ⁽¹⁴⁾SD = 0.14; ⁽¹⁵⁾SD = 0.32;
⁽¹⁶⁾SD = 2.19; ⁽¹⁷⁾SD = 3.11; ⁽¹⁸⁾SD = 5.02
N = Number of mice

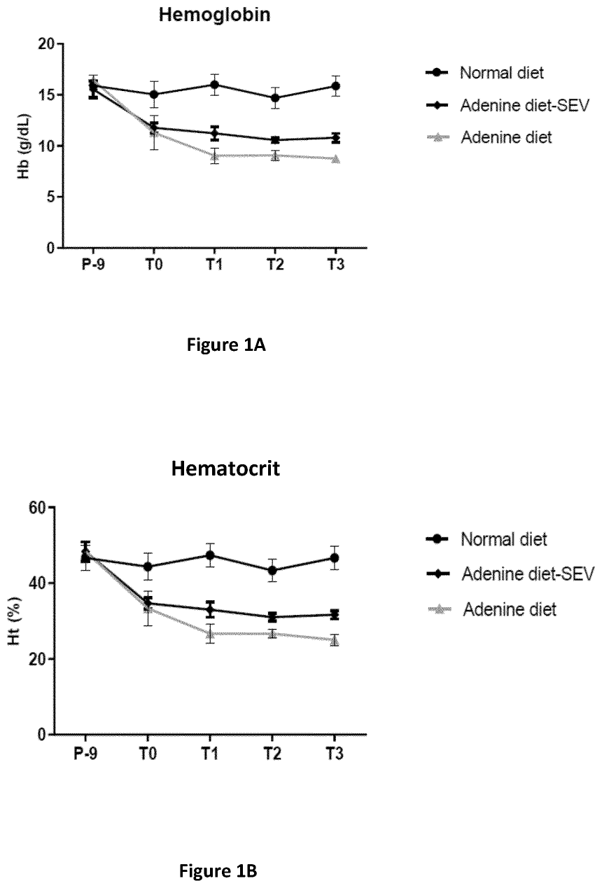

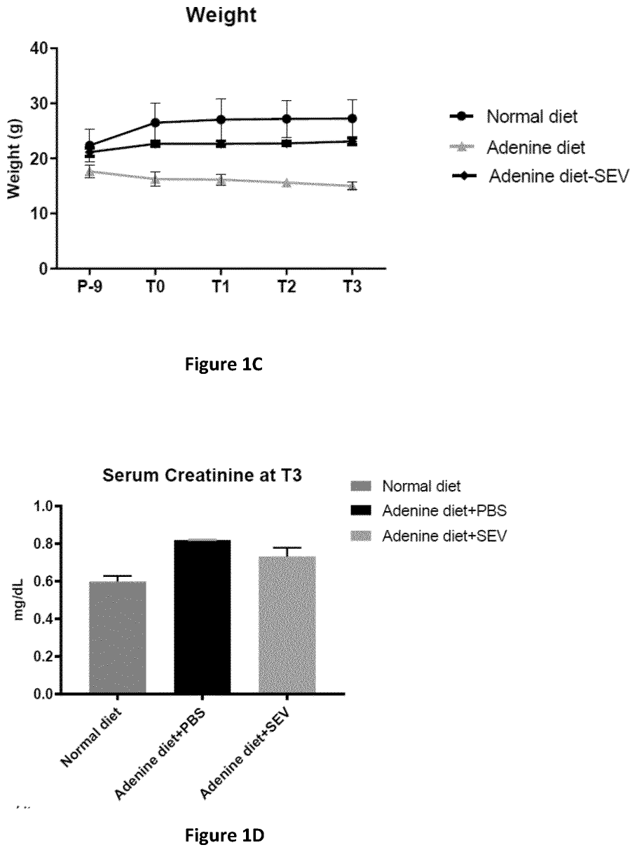

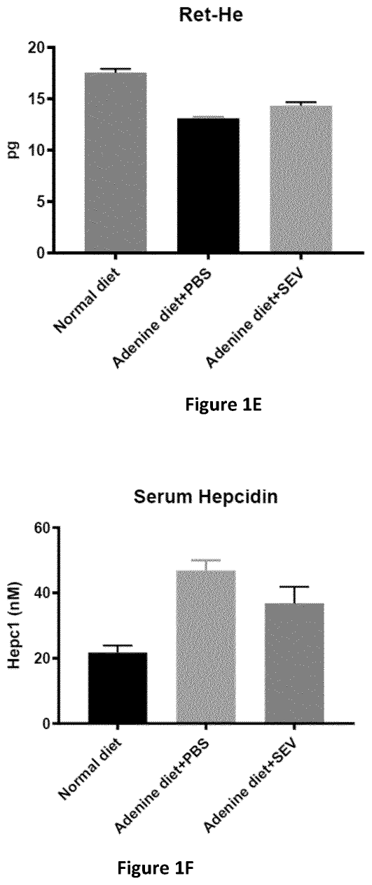

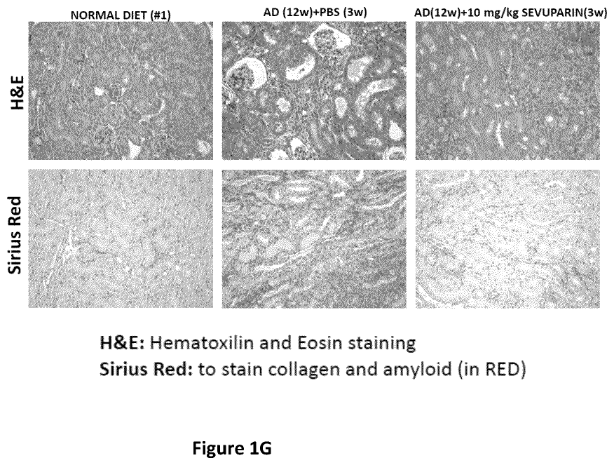

A shown in Table 1A, the following could be concluded:
(i) Hemoglobin blood levels increased in mice treated with sevuparin for 3 weeks compared to mice which had not been treated with sevuparin. These results are also shown in FIG. 1A.
(ii) Hematocrit levels increased in mice treated with sevuparin for 3 weeks compared to mice which had not been treated with sevuparin. These results are also shown in FIG. 1B.
(iii) Mice treated with sevuparin for 3 weeks gained body weight compared to mice which had not been treated with sevuparin. These results are also shown in FIG. 1C.
(iv) The serum creatinine level for mice treated with sevuparin for 3 weeks increased compared to mice which had not been treated with sevuparin. These results are also shown in FIG. 1D.
(v) The blood level of reticulocyte-hemoglobin (Ret-He) in mice treated with sevuparin for 3 weeks increased compared to mice which had not been treated with sevuparin. These results are also shown in FIG. 1E.
(vi) The serum level of hepcidin in mice treated with sevuparin for 3 weeks was reduced compared to mice which had not been treated with sevuparin. These results are also shown in FIG. 1F.

Also, histochemistry and immunostaining on paraffin-embedded kidney sections were analyzed as described above. The physical status of the rat kidneys are shown in FIG. 1G (20× magnification), and as seen in this figure, sevuparin had a positive effect on the micrographs of the mouse kidneys compared to mouse kidneys without treatment with sevuparin.

### Example 2

ARD Pre-Treatment for 9 Weeks Followed by 2 Weeks Treatment with Either Control, Sevuparin, Erythropoietin (EPO) or a Combination of EPO and Sevuparin, Followed by 7 Weeks Recovery without ARD but with Maintained Treatments.

C57BL/6J male mice were exposed to ARD pretreatment for 9 weeks followed by 2 weeks of randomized allocation to treatments as defined below still with maintained ARD exposure, after which ARD was omitted but treatments were maintained during 7 weeks of recovery. One group of mice was given normal diet (no ARD) for the entire experiment, as healthy control.
a) Treatment with control: mice treated with PBS s.c. every day for 2+7 weeks.
b) Treatment with sevuparin: mice treated with 10 mg/kg Sevuparin s.c. every day for 2+7 weeks.
c) Treatment with sevuparin: mice treated with 50 IU EPO i.p. twice a week for 2+7 weeks.
d) Treatment with EPO+sevuparin: mice treated with 50 IU EPO i.p. twice a week and 10 mg/kg Sevuparin s.c. every day for 2+7 weeks.

Hemoglobin (Hb), hematocrit (Ht), weigh (wt), serum-creatitine (S-crea), reticulocyte hemoglobin (Ret-He) content and serum-hepcidin (nM) was measured in the mice. The results are shown in Table 2A below:

TABLE 2A

| | | | | | ARD |
|---|---|---|---|---|---|
| | Normal | ARD-PBS | | | EPO- |
| | diet | (control) | ARD-SEV | ARD-EPO | SEV |
| | N = 4 | N = 5 | N = 4 | N = 4 | N = 5 |
| Hb [g/dL] | 14.95⁽¹⁾ | 7.82⁽²⁾ | 9.45⁽³⁾ | 8.48⁽⁴⁾ | 9.22⁽⁵⁾ |
| Ht [%] | 44.0⁽⁶⁾ | 23.00⁽⁷⁾ | 27.75⁽⁸⁾ | 24.75⁽⁹⁾ | 27.20⁽¹⁰⁾ |
| Wt [g] | 29.40⁽¹¹⁾ | 25.08⁽¹²⁾ | 25.75⁽¹³⁾ | 26.43⁽¹⁴⁾ | 26.40⁽¹⁵⁾ |
| S-Crea [mg/dL] | 0.68⁽¹⁶⁾ | 0.95⁽¹⁷⁾ | 0.78⁽¹⁸⁾ | 0.76⁽¹⁹⁾ | 0.66⁽²⁰⁾ |
| Ret-He [pg] | 17.2⁽²¹⁾ | 14.82⁽²²⁾ | 15.78⁽²³⁾ | 14.75⁽²⁴⁾ | 15.06⁽²⁵⁾ |
| S-Hepcidin [nM] | 11.72⁽²⁶⁾ | 49.06⁽²⁷⁾ | 22.32⁽²⁸⁾ | 22.25⁽²⁹⁾ | 42.29⁽³⁰⁾ |

⁽¹⁾SD = 0.44; ⁽²⁾SD = 1.13; ⁽³⁾SD = 0.79; ⁽⁴⁾SD = 0.1.14; ⁽⁵⁾SD = 0.68;
⁽⁶⁾SD = 1.41; ⁽⁷⁾SD = 3.46: ⁽⁸⁾SD = 2.36; ⁽⁹⁾SD = 3.50; ⁽¹⁰⁾SD = 2.17;
⁽¹¹⁾SD = 1.20; ⁽¹²⁾SD = 1.03; ⁽¹³⁾SD = 0.82; ⁽¹⁴⁾SD = 2.88; ⁽¹⁵⁾SD = 0.54;
⁽¹⁶⁾SD = 0.17; ⁽¹⁷⁾SD = 0.19; ⁽¹⁸⁾SD = 0.09; ⁽¹⁹⁾SD = 0 .05; ⁽²⁰⁾SD = 0.11;
⁽²¹⁾SD = 0.22; ⁽²²⁾SD = 0.64; ⁽²³⁾SD = 0.22; ⁽²⁴⁾SD = 0.52; ⁽²⁵⁾SD = 0.40;
⁽²⁶⁾SD = 1.25; ⁽²⁷⁾SD = 20.21; ⁽²⁸⁾SD = 9.01; ⁽²⁹⁾SD = 5.94; ⁽³⁰⁾SD = 14.95.
N = Number of mice

A shown in Table 2A, the following could be concluded:
(i) The blood level of hemoglobin (Hb) was increased in mice treated with a combination of EPO and sevuparin, compared to mice without any treatment, and for mice treated with EPO as monotherapy. The combination treatment EPO+sevuparin was better than monotherapy with EPO, but combination treatment EPO+sevuparin was slightly inferior compared to monotherapy with sevuparin.
(ii) The hematocrit (Ht) was increased in mice treated with sevuparin compared to mice without any treatment, and for mice treated with EPO as monotherapy. The combination treatment EPO+sevuparin was better than monotherapy with EPO, but combination treatment EPO+sevuparin was slightly inferior compared to monotherapy with sevuparin.
(iii) The body weight (wt) was increased in mice treated with sevuparin compared to mice without any treatment, even though mice treated with EPO as monotherapy gained slightly more weight. Mice treated with the combination of EPO+sevuparin gained more weight than mice receiving monotherapy with sevuparin.
(iv) The serum level of creatinine (s-Crea) was reduced in mice treated with sevuparin compared to mice without any treatment. The serum level of creatinine was also reduced in mice treated with EPO as monotherapy, even though not as much as for monotherapy with sevuparin. Combination therapy with EPO+sevuparin was even more beneficial with regard to the serum level of creatinine.
(v) The blood level of reticulocyte hemoglobin (Ret-HE) was increased in mice treated with sevuparin compared to mice without any treatment. However, monotherapy with EPO as well as combination therapy with EPO+sevuparin, was inferior to monotherapy with sevuparin.
(vi) The serum level of hepcidin was markedly reduced in mice treated with sevuparin as monotherapy, as well as mice treated with EPO as monotherapy. Combination therapy with EPO+sevuparin did however increase the serum level of hepcidin.

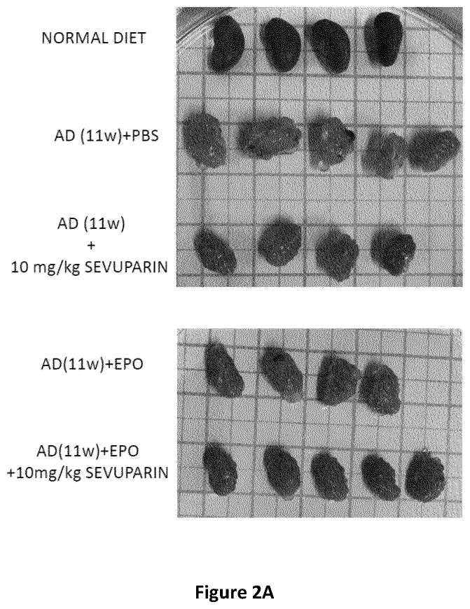

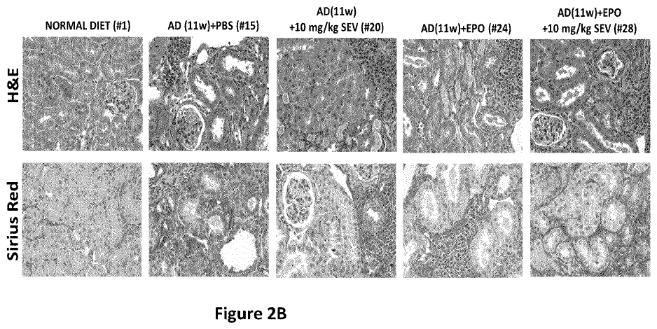

The physical status of the mouse kidneys were also investigated, which is shown in FIG. 2A. Macroscopic photographs of mouse kidneys treated with sevuparin, or with a combination of sevuparin and EPO, have substantially less cysts, more normal colour and a more homogenous appearance compared to mouse kidneys that had not received the same therapy.

Moreover, as shown in FIG. 2B, the histological analysis of the mouse kidneys confirms the above macroscopic observations (40× magnification): the adenine diet caused a profound damage of the organ after 11 weeks of adenine diet, compared to the normal diet group. The H&E staining showed features of tubulointerstitial damage and adenine crystal deposition in the tissue from adenine treated animals. The treatment with sevuparin as well as the treatment with EPO reduced the damage, compared to the untreated control.

The Sirius Red Staining in FIG. 2B (40× magnification) show the deposits of collagen after adenine diet indicated by the red fibers, demonstrating fibrosis. Treatment with Sevuparin showed a reduction of the collagen deposits indicative of reduced fibrosis in the kidney tissue, compared to the group treated with PBS, with sevuparin, or with a combination of EPO and sevuparin (AD 11 weeks+PBS).

This experiment indicates that treatment with sevuparin alone, as well as with a combination of sevuparin and EPO, protects the kidneys from kidney damage and fibrosis. This in turn reduces the risk for kidney failure.

### Example 3

ARD Pretreatment for 5.5 Weeks Followed by 3 or 6 Weeks Treatment with Control, Sevuparin, Erythropoietin (EPO), or a Combination of EPO and Sevuparin

C57BL/6J male mice were exposed to ARD pretreatment for 5.5 weeks followed by either 3 or 6 weeks of randomized allocation to treatments as defined below still with maintained ARD exposure. One group of mice was given normal diet (no ARD) for the entire experiment, as healthy control.
a) Treatment control: mice treated with PBS s.c. every day for 3 and 6 weeks respectively.
b) Treatment with sevuparin: mice treated with 10 mg/kg Sevuparin s.c. every day for 3 and 6 weeks respectively.
c) Treatment with EPO: mice treated with 50 IU EPO i.p. twice a week for 3 weeks and mice treated with 50 IU EPO i.p. twice a week for 3 weeks followed by treatment with 25 IU EPO i.p. once a week for the subsequent 3 weeks.
d) Treatment with EPO+sevuparin: mice treated with 10 mg/kg sevuparin s.c. every day and 50 IU EPO i.p. twice a week for 3 weeks and mice treated with 10 mg/kg sevuparin s.c. every day and 50 IU EPO i.p. twice a week for the first 3 weeks followed by treatment with 10 mg/kg sevuparin s.c. every day and 25 IU EPO i.p. once a week for the subsequent 3 weeks.

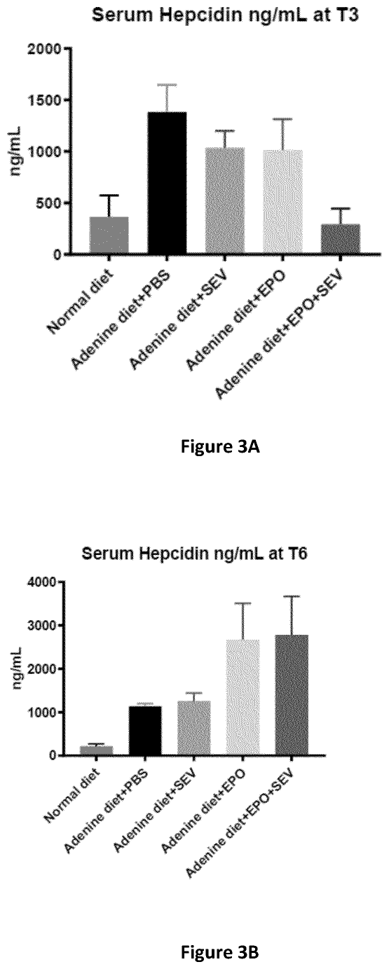

Hemoglobin (Hb), hematocrit (Ht), weigh (wt), serum-creatitine (S-crea), reticulocyte hemoglobin (Ret-He) content and serum-hepcidin (nM) was measured in the mice. The results are shown in Table 3A below and in FIG. 3A and FIG. 3B:

TABLE 3A

| | Normal | ARD-PBS | | | ARD |
|---|---|---|---|---|---|
| | diet | (control) | ARD-SEV | ARD-EPO | EPO-SEV |
| | N = 3 | N = 3 | N = 3 | N = 4 | N = 4 |
| Hb [g/dL] | 15.73⁽¹⁾ | 10.07⁽²⁾ | 9.77⁽³⁾ | 9.78⁽⁴⁾ | 18.45⁽⁵⁾ |
| Ht [%] | 46.33⁽⁶⁾ | 29.33⁽⁷⁾ | 28.67⁽⁸⁾ | 28.75⁽⁹⁾ | 54.25⁽¹⁰⁾ |
| Wt [g] | 27.93⁽¹¹⁾ | 16.50⁽¹²⁾ | 17.60⁽¹³⁾ | 16.33⁽¹⁴⁾ | 19.45⁽¹⁵⁾ |
| S-Crea [mg/dL] | 0.57⁽¹⁶⁾ | 0.84⁽¹⁷⁾ | 0.82⁽¹⁸⁾ | 0.99⁽¹⁹⁾* | 0.81⁽²⁰⁾ |
| Ret-He [pg] | 17.33⁽²¹⁾ | 14.70⁽²²⁾ | 14.45⁽²³⁾** | 12.83⁽²⁴⁾ | 15.62⁽²⁵⁾ |

⁽¹⁾SD = 0.76; ⁽²⁾SD = 0.68; ⁽³⁾SD = 2.35; ⁽⁴⁾SD = 2.88; ⁽⁵⁾SD = 2.85;
⁽⁶⁾SD = 2.08; ⁽⁷⁾SD = 2.08; ⁽⁸⁾SD = 7.02; ⁽⁹⁾SD = 8.38; ⁽¹⁰⁾SD = 8.22;
⁽¹¹⁾SD = 1.44; ⁽¹²⁾SD = 2.91; ⁽¹³⁾SD = 1.97; ⁽¹⁴⁾SD = 3.29; ⁽¹⁵⁾SD = 0.44;
⁽¹⁶⁾SD = 0.032; ⁽¹⁷⁾SD = 0.045; ⁽¹⁸⁾SD = 0.064; ⁽¹⁹⁾SD = 0.19; ⁽²⁰⁾SD = 0.036; *N = 3
⁽²¹⁾SD = 0.25; ⁽²²⁾SD = 0.36; ⁽²³⁾SD = 0.64; ⁽²⁴⁾SD = 0.29; ⁽²⁵⁾SD = 0.95; **N = 2.
N = Number of mice

A shown in Table 3A, the following could be concluded:
(i) The blood level of hemoglobin (Hb) was increased in mice treated with a combination of EPO and sevuparin, whereas monotherapy with sevuparin and EPO respectively, did not increase the blood level of hemoglobin.
(ii) The hematocrit (Ht) was increased in mice treated with a combination of EPO and sevuparin, whereas monotherapy with sevuparin and EPO respectively, did not increase the blood level of hematocrit.
(iii) The body weight (wt) was increased in mice treated with a combination of EPO and sevuparin, whereas monotherapy with sevuparin and EPO respectively, did not increase the body weight.
(iv) The serum level of creatinine (s-Crea) was reduced in mice treated with sevuparin compared to mice without any treatment. The serum level of creatinine was also reduced in mice treated combination therapy with EPO+sevuparin, but monotherapy with EPO increased the serum level of creatinine.
(v) The blood level of reticulocyte hemoglobin (Ret-HE) was increased in mice treated with combination therapy with EPO+sevuparin.

The serum level of hepcidin was also measured, at 3 weeks treatment as well as at 6 weeks treatment, and the results are shown in FIG. 3A and FIG. 3B. As seen in the graph at 3 weeks (FIG. 3A), sevuparin monotherapy, EPO monotherapy, as well as combination therapy had a beneficial impact on the serum level of hepcidin. However, at 6 weeks treatment (FIG. 3B), the beneficial impact on the serum level of hepcidin vs control was no longer present, while the benefit on anemia for combination therapy with EPO+sevuparin remained stable indicating that by this time the combination could treat the anemia without direct involvement of a decreasing effect on hepcidin. This is further supported by the maintained high Ret-He value in the group with combination therapy with EPO+sevuparin whereas this had dropped in the group with monotherapy EPO, indicating less functional availability of iron and thereby less efficient erythropoiesis.

1-26. (canceled)

27. A method for the treatment of a chronic kidney disease (CKD), comprising administering a therapeutically effective amount of the compound sevuparin or a pharmaceutically acceptable salt thereof, to a subject in need of such treatment.

28. The method of claim 27, wherein the chronic kidney disease (CKD) comprises kidney damage.

29. The method of claim 28, wherein the kidney damage comprises inflammation.

30. The method of claim 28, wherein the chronic kidney disease (CKD) comprises fibrosis.

31. The method of claim 27, wherein the chronic kidney disease (CKD) comprises an impaired kidney function with a CKD glomular filtration rate (GFR) which is G2, G3a, G3b, or G4, as defined by the National Kidney Foundation.

32. The method of claim 27, wherein a subject suffering from the chronic kidney disease (CKD) is at risk of developing kidney failure.

33. The method of claim 27, wherein the subject suffering from the chronic kidney disease (CKD) has kidney failure.

34. The method of claim 27, wherein the chronic kidney disease (CKD) comprises anemia.

35. The method of claim 34, where the anemia is anemia in chronic disease (ACD).

36. The method of claim 34, wherein the anemia is renal anemia.

37. The method of claim 27, wherein the sevuparin is used in combination therapy with an agent for use as standard of care (SOC) therapy for chronic kidney disease (CKD).

38. The method of claim 37, wherein an erythropoiesis-stimulating agent (ESA) is used as standard of care (SOC) therapy.

39. The method of claim 38, wherein the erythropoiesis-stimulating agent (ESA) is an erythropoietin (EPO).

40. The method of claim 39, wherein the erythropoietin (EPO) is selected from the group consisting of erythropoietin alfa; erythropoietin beta; erythropoietin epsilon; erythropoietin gamma; erythropoietin kappa; erythropoietin omega; erythropoietin theta; and erythropoietin zeta.

41. The method of claim 37, wherein a HIF prolyl hydroxylase inhibitor is used as standard of care (SOC) therapy.

42. The method of claim 41, wherein the HIF prolyl hydroxylase inhibitor is selected from roxadustat, vadadustat, dapurodustat, enarodustat, and molidustat.

43. The method of claim 37, wherein the combination therapy is add-on therapy.

44. The method of claim 37, wherein the compound sevuparin and the agent used as standard of care (SOC) therapy are co-administered.

45. The method of claim 37, wherein the compound sevuparin and the agent used as standard of care (SOC) therapy are administered simultaneously, separately or sequentially.

46. The method of claim 27, wherein said use is in a subject where monotherapy with an erythropoiesis-stimulating agent (ESA) does not provide adequate therapeutic effect.

47. The method of claim 27, wherein the therapeutic effect of sevuparin does not depend on the serum level of hepcidin.

48. The method of claim 27, wherein said use is for restoring responsiveness to erythropoietin (EPO) in the subject having anemia.

49. The method of claim 27, wherein the chronic kidney disease (CKD) is present in an elderly subject.

50. The method of claim 27, wherein the anemia is present in an elderly subject.

51. The method of claim 27, wherein said use is for increasing the serum level of haemoglobin.
